(19) United States
(12) Patent Application Publication
(10) Pub. No.: US 2018/0093056 A1
(43) Pub. Date: Apr. 5, 2018

(54) MEDICO-SURGICAL TUBE ASSEMBLIES

(71) Applicant: SMITHS MEDICAL INTERNATIONAL LIMITED, Ashford (GB)

(72) Inventor: Richard Hingley, Hythe (GB)

(73) Assignee: SMITHS MEDICAL INTERNATIONAL LIMITED, Ashford (GB)

(21) Appl. No.: 15/562,465

(22) PCT Filed: Mar. 24, 2016
(86) PCT No.: PCT/GB2016/000059

§ 371 Date: Sep. 28, 2017

(30) Foreign Application Priority Data
Apr. 29, 2015 (GB) 1507548.4

(51) Int. Cl. A61M 16/04

(52) U.S. Cl. CPC A61M 16/0402; A61M 16/0463; A61M 16/0465; A61M 16/0488

(57) ABSTRACT

A tracheostomy tube assembly has a tube (1) and a flange assembly (2) that can be moved along the machine end region (10) of the tube. The flange assembly has a screw clamp (36) that can be tightened to apply a compressive force to the outside of the tube to lock the flange assembly in position. The machine end region of the tube has a non-circular section that is wider along the axis (15) along which the screw clamp applies a force. In this way, when the screw clamp (36) is tightened to lock the flange assembly (2) in position the tube (1) in the region of the clamp is deformed to a more circular shape. This avoids gripping any inner cannula (20) within the tube (1).

This invention relates to medico-surgical tube assemblies of the kind including a tube and a flange assembly movable along at least a region along the length of the tube, the flange assembly including a locking arrangement effective to apply a compressive force across the width of the tube to grip the tube and resist displacement of the flange assembly.

Medico-surgical tubes, such as tracheostomy tubes, are commonly provided with a flange to secure the tube to the patient's body. In the case of a tracheostomy tube, the flange is positioned close to the surface of the neck where the tube enters the tracheostomy, a tape is threaded through openings in the flange and fastened around the neck. For most patients, a comfortable fit can be achieved using one of a range of several different size tubes, each having a flange mounted at a fixed location along the tube suitable for patients having an average anatomy. There are, however, some situations where a fixed flange is not suitable, such as, for example, in obese patients where tissue between the neck surface and the trachea is very thick. In these situations, it is preferable for the flange to be movable along the tube to the ideal position and to be lockable in that position. Tubes with adjustable flanges are described in, for example, U.S. Pat. No. 5,026,352, U.S. Pat. No. 4,249,529, U.S. Pat. No. 4,449,527, U.S. Pat. No. 4,498,903, U.S. Pat. No. 4,530,354, U.S. Pat. No. 4,530,354, U.S. Pat. No. 4,649,913, U.S. Pat. No. 4,683,882, U.S. Pat. No. 4,774,944, WO80/02645, WO84/03217, U.S. Pat. No. 4,278,081, WO06/087513 and PCT/GB2015/000237. U.S. Pat. No. 8,104,476 describes a tube with an adjustable flange having two halves that clamped about the outside of a tube when a lever is folded flat. It is important to achieve a secure fastening of the flange to the tube even when this is wet and slippery. Because of this the flange must exert a relatively high frictional force on the outside of the tube. This can cause a problem in that the outside of the tube may be compressed, causing a localised reduction in the diameter and cross-sectional area of the bore through the tube. Where an inner cannula is used the deformation of the tube caused by the locking flange can reduce the clearance between the inside of the shaft and outside of the inner cannula to the extent that it can be difficult to remove and replace the inner cannula.

It is an object of the present invention to provide an alternative medico-surgical tube assembly.

According to the present invention there is provided a medico-surgical tube assembly of the above-specified kind, characterised in that the tube, at least in a major part of the region along which the flange assembly can be positioned during use, has an internal dimension that is wider along the axis along which the compressive force is applied than at right angles to that axis so that compression of the tube along the axis tends to reduce the difference between the internal dimension of the tube along the axis and at right angles to the axis in the region of the locking arrangement.

The interior of the tube preferably has along the major part of the region a cross-section resembling a stretched circle. The tube preferably has along the major part of the region the same internal and external section. The internal dimension of the tube along the region is preferably wider along an axis orthogonal to a plane of curvature of the tube. The locking arrangement may include a screw clamp. The assembly may include an inner cannula extending along the inside of the tube. The inner cannula preferably has a substantially circular internal and external cross section. The tube is preferably a tracheostomy tube having a patient end adapted for location within the trachea and an opposite machine end adapted for location externally of the patient, the region along which the flange assembly can be positioned being towards the machine end of the tube.

A tracheostomy tube assembly according to the present invention will now be described, by way of example, with reference to the accompanying drawings, in which:

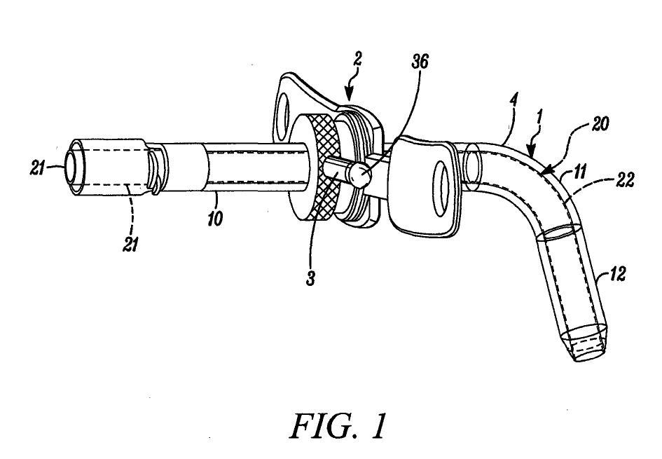

FIG. 1 is a perspective view of the assembly;

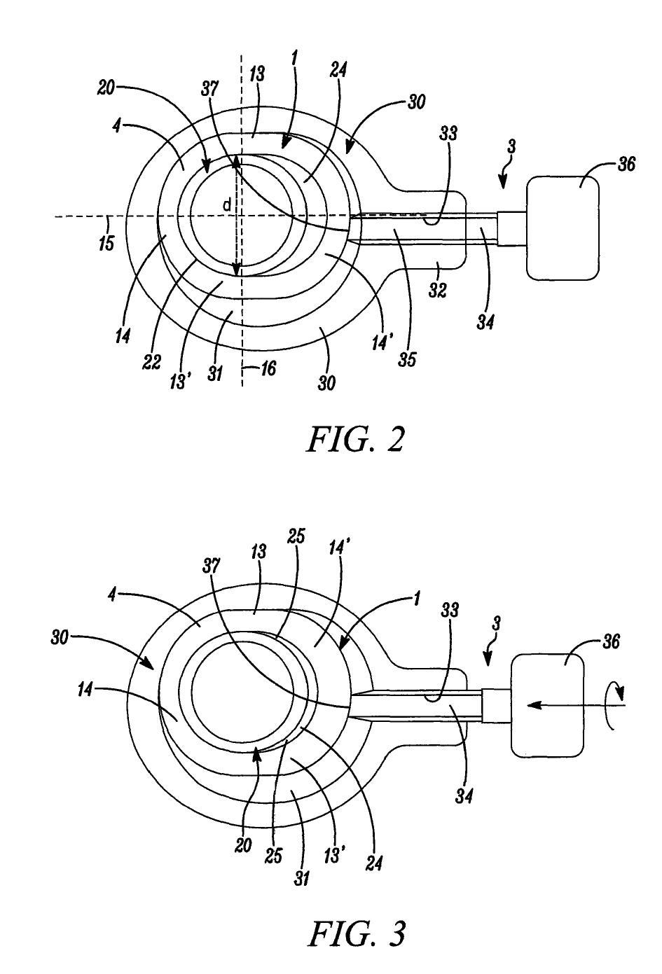

FIG. 2 is a cross-sectional view along the axis of the tube with the flange assembly in an unlocked state; and

FIG. 3 is a cross-sectional view along the axis of the tube with the flange assembly in a locked state.

With reference first to FIG. 1, the tracheostomy tube assembly comprises a tube 1 and a flange assembly 2 with a locking arrangement 3, the flange assembly being movable along the machine end region 10 of the tube and lockable at different locations along this region.

The tube 1 is made of a conventional, bendable plastics material, such as PVC, polyurethane or silicone, is hollow with a smooth inner and outer surface. The tube 1 is illustrated as not having any sealing cuff but it will be appreciated that the invention could be applied to a tracheostomy tube with an inflatable or other form of conventional sealing cuff. Similarly, the tube could have other conventional features, such as provision for suctioning above a cuff, fenestrations to enable speech and the like. The shaft 4 of the tube 1 comprises the machine end region 10, which is straight and is adapted to have its free end located externally of the patient. The shaft 4 also has a curved intermediate region 11 and a straight patient end region 12 extending at substantially 120° to the machine end region and adapted to locate in the trachea. Alternative shape shafts are possible, such as shafts that are curved continuously along their length or shafts that have a natural straight shape but are highly flexible so that they can conform readily to the shape of the anatomy. The tube 1 differs from conventional tubes in that it has a non-circular cross-section both internally and externally along at least a major part of that region of the tube along which the flange assembly 2 can be displaced and locked, that is, along at least a major part of the machine end region 10. More particularly, as shown in FIGS. 2 and 3, the shaft 4 of the tube 1 in section, along this region 10 resembles a stretched out circle having two parallel, opposite, straight sections or sides 13 and 13′ linked at opposite ends by two opposite semicircular sections 14 and 14′. This gives the tube 1 in cross section a major axis 15, extending parallel to and midway between the two parallel sides 13 and 13′, and a minor axis 16 extending orthogonally to the major axis midway between the semicircular sections 14 and 14′. The major axis 15 is longer than the minor axis 16, between their respective boundaries at both the inner and outer edges of the tube 1. The major axis 15 and the wider dimension of the tube along the machine end region 10 lies orthogonal to the plane of curvature of the tube 1. The curved region 11 of the tube and its patient end region 12 may have the same cross section shape as the machine end 10 but preferably have a circular internal and external section with an internal and external diameter the same as the internal and external diameters of the semicircular sections 14 and 14′ along the machine end region.

The tube also includes an inner cannula 20 of conventional construction having a hub 21 at the machine end of a flexible shaft 22 of circular section. The external diameter of the shaft 22 is substantially the same as, or slightly less than, the internal diameter of the tube 1 along its regions 11 and 12, having a circular section and is the same as or slightly less than the internal diameter “d” of the semi-circular sections 14 and 14′ along the machine end region 10. The wall of the shaft 22 is as thin as possible so that its inner bore is as large as possible and the inner cannula 20 provides as little impedance to gas flow along the tube 1 as possible. The wall of the shaft 22, however, must be sufficiently strong to resist buckling during insertion into the tube 1. It can be seen from FIG. 2 that, when the locking flange 2 is unlocked, the inner cannula 20 will contact, or be closely adjacent to, the straight sides 13 and 13′ of the tube 1 along the region 10 but that there will be a crescent-shape gap 24 on one or both sides of the inner cannula between the outside of the cannula and the inside of the or each of the semi-circular sides 14 and 14′.

The tube 1 could be made in various different ways. For example, it could be extruded with a constant circular section and its machine end region subsequently formed with the desired section by heating to soften and inserting a mandrel of the appropriate shape. Alternatively, the tube could be moulded individually in a mould tool or cast on a mandrel of the appropriate section. Another manufacturing technique could involve making the tube in two halves of differing cross-sections that are subsequently joined together end-to-end. In a further technique the tube would initially be made, such as by extrusion, with the stretched circular section along its entire length and the patient and intermediate regions subsequently formed to a conventional circular shape such as by heating and compressing in between an inner core pin and an outer mould. Further alternative techniques could involve, for example, 3-D printing techniques.

The locking arrangement 3 of the flange 2 takes the form of a clamp that bears on the outside of the tube 1 and, more particularly, a clamp that applies pressure across the width of the tube (along the major axis 15), between opposite sides of the tube, to grip the tube. The locking arrangement 3 illustrated includes an outer collar 30 extending around the outside of the tube 1 and having an internal diameter larger than the external diameter of the tube, thereby leaving a gap 31 between the outside of the tube and the inside of the collar. At one side, the collar 30 is formed with a radially outwardly-extending cylindrical boss 32 having an axial, screw-threaded bore 33 extending through it. The boss 32 is aligned with the length of the flange 2, that is, orthogonally to the plane of curvature of the tube 1 and parallel to the straight sections 13 and 13′ in the machine end region 10 of the tube. A clamping member in the form of a screw 34 has a threaded shaft 35 in threaded engagement in the bore 33 of the boss 32 and has an enlarged head 36 at its outer end for manual engagement by which the screw can be screwed into or out of the boss 32. The inner end 37 of the screw 34 is movable into contact with the outside of the tube 1.

In the state shown in FIG. 2, where the locking arrangement 3 is in an unlocked state, the inner end 37 of the screw 34 only contacts the tube 1 lightly so as to allow the locking arrangement 3 and connected flange 2 to slide freely along the straight, machine end region 10 of the tube so that it can be positioned as desired. In this state, the inner end 37 of the screw 34 might be entirely out of contact with the tube. When the flange 2 has been moved to the desired position, the locking arrangement 3 is engaged to lock it in position. This is done by gripping the head 36 of the screw 34 and twisting it clockwise so that the screw is moved inwardly of the boss 32 (to the left in FIGS. 2 and 3) and its inner end 37 applies a compressive force across the tube 1 parallel with the major axis 15 and the straight sides 13 and 13′. This force squeezes and grips the tube 1 laterally so that its cross-section in the region of the locking arrangement 3 changes from the shape shown in FIG. 2 to the shape shown in FIG. 3 where the semicircular side 14′ contacted by the screw 34 has been moved inwardly towards the inner cannula 20. This lateral squeezing inwards also has the effect of displacing the straight sections 13 and 13′ outwardly (Poisson effect) to a curved shape so that the tube 1 in this region adopts a cross section that is closer to being completely circular. So, although the clearance or gap 24 between the right-hand side of the inner cannula 20 and the inside of the semicircular section 14′ has been reduced, the clearance 25 between the upper and lower sides of the inner cannula and the previously straight sections 13 and 13′ has been increased slightly. It can be seen, therefore, that, even though the screw 34 applies a relatively high force to the tube 1, sufficient to deform the tube and apply a high friction force that locks the flange 2 in position on the tube, the inside of the tube does not compress or deform the inner cannula 20. This ensures that the gas passage along the bore of the inner cannula 20 is maintained and also ensures that the inner cannula can be pulled out of the tube 1 and replaced when necessary without having to release the lock 3 on the flange 2.

The present invention reduces the need to reinforce the tube to resist deformation, which can add to the cost of the tube and which can require the wall of the tube to be thickened, resulting in a reduced gas passage along the tube and increased stiffness.

The non-circular section of the tube along the machine end 10 of the tube has the added advantage of reducing the tendency of the tube to twist along this region. This helps ensure that the flange 2 remains correctly oriented relative to the curve of the tube and hence to the trachea. If the tube needs to have a lumen within its wall thickness, such as a cuff inflation lumen, or a suction lumen, this could be displaced away from the clamp screw, such as along the straight sections 13 or 13′ to avoid the lumen being occluded. Alternatively, the lumen could be aligned with the screw clamp providing the end of the clamp that contacted the tube was shaped to reduce the risk of occlusion. This could be achieved by a saddle-shape termination at the end of the clamp arranged with portions that engage the tube on either side of the lumen. The non-circular section of the machine end of the tube could be present on just the interior, with the external section being circular.

The locking arrangement need not have a screw clamp but could have other kinds of clamp, such as including a cam member, lever or other device.

It is not essential that the tube include an inner cannula since the invention would also have advantages in ensuring free passage of other items along the bore of a tube with a clamped locking flange, such as a suction catheter or endoscope.

The invention is not confined to tracheostomy tubes but could be used with other medico-surgical tubes having a movable and lockable flange for supporting the tube where it emerges from the body.

1-8. (canceled)

9. A medico-surgical tube assembly including a tube and a flange assembly movable along at least a region along the length of the tube, the flange assembly including a locking arrangement effective to apply a compressive force across the width of the tube to grip the tube and resist displacement of the flange assembly, characterised in that the tube, at least in a major part of the region along which the flange assembly can be positioned during use, has an internal dimension that is wider along the axis along which the compressive force is applied than at right angles to that axis so that compression of the tube along the axis tends to reduce the difference between the internal dimension of the tube along the axis and at right angles to the axis in the region of the locking arrangement.

10. An assembly according to claim 9, characterised in that the interior of the tube has along the major part of the region a cross-section resembling a stretched circle.

11. An assembly according to claim 9, characterised in that the tube has along the major part of the region the same internal and external section.

12. An assembly according to claim 9, characterised in that internal dimension of the tube along the region is wider along an axis orthogonal to a plane of curvature of the tube.

13. An assembly according to claim 9, characterised in that the locking arrangement includes a screw clamp.

14. An assembly according to claim 9, characterised in that the assembly includes an inner cannula extending along the inside of the tube.

15. An assembly according to claim 14, characterised in that the inner cannula has a substantially circular internal and external cross section.

16. An assembly according to claim 9, characterised in that the tube is a tracheostomy tube having a patient end adapted for location within the trachea and an opposite machine end adapted for location externally of the patient, and that the region along which the flange assembly can be positioned is towards the machine end of the tube.
